(19) United States
(12) Patent Application Publication
(10) Pub. No.: US 2020/0138764 A1
(43) Pub. Date: May 7, 2020

(54) METHODS AND DEVICES FOR ACTIVATING ADIPOSE TISSUE

(71) Applicant: The Procter & Gamble Company, Cincinnati, OH (US)

(72) Inventors: Jose Mauro Goulart BRUM, Loveland, OH (US); Nicholas William GEARY, Cincinnati, OH (US); John Christian HAUGHT, Argyle, TX (US); Bhavani KASIBHATLA, West Chester, OH (US); Leo Timothy LAUGHLIN, II, Mason, OH (US); Koti Tatachar SREEKRISHNA, Mason, OH (US); John August WOS, Mason, OH (US); Kenneth Edward YELM, Hamilton, OH (US)

(21) Appl. No.: 16/676,479

(22) Filed: Nov. 7, 2019

Related U.S. Application Data
(60) Provisional application No. 62/756,673, filed on Nov. 7, 2018.

(51) Int. Cl. A61K 31/215; A61K 31/165; A61P 3/04

(52) U.S. Cl. CPC A61K 31/215; A61P 3/04; A61K 31/165

(57) ABSTRACT

Treatment for excess adipose tissue by applying an activating compound directly to a targeted area. The activating compound is a cyclohexanecarboxamide derivative. The activating compound promotes thermogenesis in cells to generate heat.

### FIELD OF THE INVENTION

The present invention relates to methods for activating adipose tissue by contacting an affected area with an activating compound, wherein the adipose tissue undergoes thermogenesis upon contact with the activating compound. This invention also relates to devices comprising a therapeutically effective amount of the activating compound.

### BACKGROUND OF THE INVENTION

Obesity has reached pandemic proportions, affecting all ages and socioeconomic groups. The World Health Organization estimated that in 2008, 1.5 billion adults aged 20 years and older were overweight and over 200 million men and 300 million women were obese. These figures are estimated to increase to 2.16 billion overweight and 1.12 billion obese individuals by 2030. Obesity is the source of lost earnings, restricted activity days, absenteeism, lower productivity at work (presenteeism), reduced quality of life, permanent disability, significant morbidity and mortality, and shortened lifespan. Indeed, the total annual economic cost of overweight and obesity in the United States and Canada due to medical costs, excess mortality, and disability was estimated to be about $300 billion in 2009. International studies on the economic costs of obesity have shown that they account for between 2% and 10% of total health care costs.

Obesity is the result of a chronic imbalance between energy intake and expenditure. This leads to the storage of excess energy as adipocytes, which typically exhibit both hypertrophy (increase in cell size) and hyperplasia (increase in cell number or adipogenesis). The rise of the obesity pandemic is due to the combination of excessive consumption of energy-dense foods high in saturated fats and sugars, and reduced physical activity.

Recently, there has been a burst of new anti-obesity drugs. The increasing interest in anti-obesity drug development reflects an evolving appreciation for the molecular intricacies of this multifaceted, chronic disease. Today's anti-obesity drugs—including the five recent approvals and several more in development—focus either on appetite suppression or the reduction of the absorption of fat in the stomach (Xenical) as a treatment mechanism. However, other pathways have been shown to play a major role in obesity. Today, there is more information and a better understanding of the complex biology of obesity. With this deep understanding, various pathways and specifically targets and receptors that are involved in the process are becoming transparent.

Adipocytes are complex cells that have multiple functions, depending on their physical location and physiological status, including storage of energy (fat), mechanical (fat pads, covering delicate organs such as eyes), and adaptive thermogenesis. In addition, it was recently shown that adipose tissue functions as a critical determinant for spatial and temporal coordination of NAD+ biosynthesis throughout the body, thus maintaining metabolic homeostasis against nutritional and environmental perturbations. Thus, adipocytes play critical roles in systemic energy and metabolic regulation. Three forms of adipocytes, white, brown and beige have been described in humans.

White adipocytes store energy and serve as major secretory and endocrine organs that secrete adipokines (e.g. leptin, adiponectin, resistin), which perform various metabolic functions. White adipocytes make up the bulk of fatty tissues in animals. White adipose tissue is the most common type of adipose tissue and is characterized by a narrow rim of cytoplasm with its nucleus pressed near the margin of the cell surrounding a single large membrane-enclosed lipid droplet and a few mitochondria, modest blood supply and serves as a depot of stored energy. Also, white adipocyte is an endocrine organ and secretes, leptin, adiponectin, and asprosin hormones that regulate various metabolic process. New adipocytes in white adipose tissue are formed throughout life from a pool of precursor cells. These are needed to replace those that die (after an average life span of 10 years). In addition to serving as a major source of energy reserves, white adipose tissue also provides some mechanical protection and insulation to the body. Obesity is the excessive accumulation of white adipose tissue.

Brown adipocytes are highly specialized cells that dissipate stored chemical energy in the form of heat. They achieve this by uncoupling protein-1 (UCP-1), a mitochondrial protein that is present in brown adipose tissue. Cold stimuli and/or certain molecules can activate UCP-1 in the existing brown adipocytes, thus increasing total energy expenditure by a magnitude proportional to the number of available brown adipocytes. Adult humans have significant depots of brown adipose tissue, and these can be activated when exposed to cold temperatures. Brown adipose tissue is a key site of heat production (thermogenesis). Brown adipose tissue is characterized by the presence of cytoplasm throughout the cell with a central nucleus, many small lipid droplets, many mitochondria, that are rich in UCP-1, and rich in blood supply. UCP-1, when activated, short circuits the electrochemical gradient that drives ATP synthesis to generate heat instead. Brown adipose tissue provides a vital source of heat to maintain body temperature. Brown adipose tissue is activated when the body temperature drops.

Beige adipocytes are cells that form from white adipocytes upon stimulation. Beige adipocytes can be found interspersed in white adipose tissue, but can express UCP-1. The UCP-1 in beige adipocytes can also be activated by cold stimuli and/or certain molecules. Beige adipocytes can be recruited or induced to form from white adipocytes. Beige adipose tissue are brown-like adipocytes derived from white fat cells after a period of vigorous exercise. After exercise, skeletal muscle cells secrete a protein hormone called irisin. Irisin acts on white adipose tissue to increase the number of adipocytes that are rich in mitochondria and lipid droplets; a marked increase in the synthesis of UCP1; an increase in the rate of cellular respiration, but with the energy released as heat rather than fueling the synthesis of ATP. Lean adult humans have deposits of beige adipocytes in the neck and upper chest regions. When exposed to cold, beige adipocytes are activated. Obese people have few or no beige cells.

Fully stimulated brown or beige adipocytes have comparable amounts of UCP-1 suggesting similar thermogenic capacity. Thus, increasing the activity of brown adipocytes, beige adipocytes, or both holds a tremendous promise for the treatment of metabolic disorders.

Adipocyte thermogenesis is the process of converting energy stored in the body into heat in organisms. There are at least three types of thermogenesis methods. The first type of thermogenesis is work-induced thermogenesis. This occurs when an organism uses its muscles to create heat through movement.

The second type of thermogenesis is thermo-regulatory thermogenesis. This type of thermogenesis produces heat to maintain an organism's body temperature through shivering. Shivering produces heat by converting the chemical energy stored in the form of ATP into kinetic energy and heat. The kinetic energy generated produces the characteristic muscle twitches associated with shivering.

The third type of thermogenesis is diet-induced thermogenesis. In diet-induced thermogenesis, a portion of dietary calories in excess of those required for immediate energy requirements are converted to heat rather than stored as adipose tissue. Some types of obesity may be related to a defect in this mechanism. Diet-induced thermogenesis includes non-shivering thermogenesis, which can occur in brown or beige adipocytes. In brown and beige adipocytes, UCP-1 starts an activation cascade, which leads to the production of heat. Non-shivering thermogenesis can be controlled by the sympathetic nervous system. The sympathetic nervous system can activate thermogenesis due to various stimuli, such as cold, the ingestion of food, and various other hormones and chemical stimuli.

Adipocyte thermogenesis and energy metabolism are reduced in obese individuals. Thus, activating brown or beige adipocytes to enhance energy expenditure is of great interest to combat obesity. In addition, conversion of existing white fat cells to brown or beige fat cells could also increase non-shivering thermogenesis and metabolism. Therefore, specific materials that stimulate brown cell development; materials that increase UCP-1 expression in various types of adipocytes; and materials that augment brown adipose tissue mass are of interest. The latter can also be increased through low temperature, hibernation and/or molecules directing brown adipocyte differentiation.

The current symptomatic medical treatments of obesity fail to achieve their long-term therapeutic goals, largely due to limited drug efficacy, side effects, and patients' poor adherence with lifestyle changes along with therapies. Presently, only restrictive and malabsorptive bariatric surgery can achieve significant long-term reduction of weight excess with some favorable cardiovascular benefits.

Accordingly, there is a need in the art for novel treatments for obesity beyond drugs that merely suppress appetite or lower fat absorption. The present invention provides methods and medical devices for the local activation of adipocytes by applying an activating compound. The activating compound activates thermogenesis in white, brown, or beige adipose tissue, which can lead to the generation of heat, lipolysis of adipose tissue, and ultimately lead to the overall reduction in quantity and size of adipose tissue.

### BRIEF DESCRIPTION OF THE FIGURE

FIG. 1 shows an example of segmentation (right) from a stained image (left). The inter-cellular region was stained as wine-red color while the adipocyte was light-yellow in an original image. Inter-cellular and adipocyte segments are shown by yellow and dark-blue.

### SUMMARY OF THE INVENTION

A method of promoting thermogenesis comprising contacting one or more adipocytes with an activating compound, wherein the activating compound comprises the following structure or salts thereof:

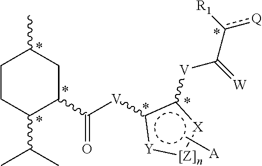

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH,
or heteroatom;
A=lower alkoxy, lower alkylthio, aryl, substituted aryl or fused aryl; and
stereochemistry is variable at the positions marked*.

A method of promoting thermogenesis comprising contacting one or more adipocytes with an activating compound, wherein the activating compound comprises the following structure or salts thereof:

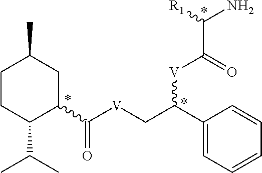

R₁ is H, alkyl, amino alkyl, or alkoxy;
V is —O— or —(NH)—; and
stereochemistry is variable at the positions marked*.

A method of promoting thermogenesis comprising contacting one or more adipocytes with an activating compound, wherein the activating compound comprises at least one of the following structures or salts thereof:

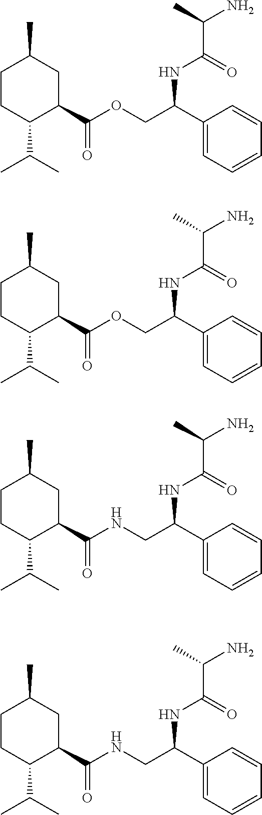

A method of treatment comprising contacting one or more adipocytes with an activating compound, wherein the activating compound comprises the following structure or salts thereof:

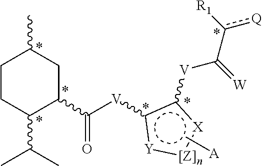

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and
stereochemistry is variable at the positions marked*.

A device is provided comprising: a therapeutically effective amount of an activating compound and a means for contacting one or more adipocytes with the activating compound.

A device is provided as described above, wherein the activating compound comprises the following structure:

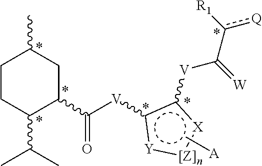

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and stereochemistry is variable at the positions marked*.

A device is provided as described above, wherein the activating compound comprises the following structure:

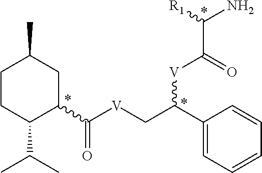

R₁ is H, alkyl, amino alkyl, or alkoxy;
V is —O— or —(NH)—; and
stereochemistry is variable at the positions marked*.

A device is provided as described above, wherein the activating compound comprises at least one of the following structures and salt thereof:

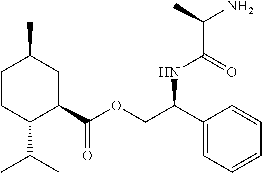 [SMILES: CC(C)[C@@H]1CC[C@@H](C)C[C@H]1C(=O)OC[C@@H](NC(=O)[C@@H](C)N)c1ccccc1]

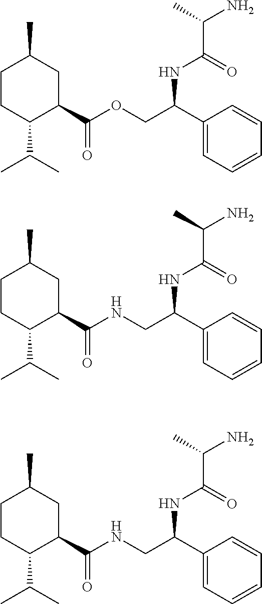

These and other features, aspects, and advantages of the present invention will become evident to those skilled in the art from the detailed description that follows.

### DETAILED DESCRIPTION OF THE INVENTION

The present invention is directed to the surprising discovery that certain cyclohexanecarboxamide derivatives (activating compounds) can activate adipose tissue to induce thermogenesis. Upon contact with one or more adipocytes or an affected area, the activating compound can promote the expression of mitochondrial proteins, including, but not limited to UCP-1, UCP-2, or combinations thereof. Expression of mitochondrial proteins, such as UCP-1, UCP-2, or combinations thereof, can activate one or more adipocytes to induce thermogenesis. White, brown, and/or beige adipocytes can be activated to induce thermogenesis upon contact with the activating compound.

The present invention is thus based on the surprising discovery that select molecules can be used to activate one or more adipocytes to induce thermogenesis. A second object of this invention shows the discovery that select molecules, such as certain cyclohexanecarboxamide derivatives or activating compounds, can treat obesity and obesity related diseases, including but not limited to type 1 diabetes, type 2 diabetes, insulin-resistance, dyslipidemia, chronic pain, neuropathic pain, inflammatory pain, and irritable bowel syndrome. Additionally, the present invention shows the surprising discovery that select molecules as described above and herein can treat obesity, reduce the size and quantity of adipose tissue, lead to body contouring, body shaping, and ultimately weight loss.

While some compounds have been previously shown to promote thermogenesis, such as in U.S. Patent Application Publication No. 2018/0147163, they have required multiple active agents, each with different mechanisms to contribute to weight loss. In contrast, disclosed herein, are compounds with unexpectedly high activity to promote thermogenesis. The activity shown by the disclosed compositions can be high enough to allow for compositions including only a single activating compound. As such, thermogenesis can be promoted with only a single activating compound due to the high activity displayed by the disclosed compositions. While not wishing being bound by theory, disclosed herein is a method and device capable of inducing brown, beige, and white adipocytes to induce thermogenesis. As described herein, non-shivering thermogenesis can be stimulated by cold temperatures. However, surprisingly, certain cooling compounds that were previously shown to activate the TRPM8 receptor in oral care compositions to provide a cooling sensation (U.S. Patent App. Pub. No. 2017-0119639, herein incorporated by reference) have also been shown to activate one or more adipocytes and/or adipose tissue to induce thermogenesis. Activation of TRMP8 and/or promotion of thermogenesis in one or more adipocytes and/or adipose tissue can lead to adipocyte differentiation (i.e. pre-adipocytes preferentially developing into brown adipocytes instead of white adipocytes) and/or the conversion of white adipocytes to beige and/or brown adipocytes.

Without wishing to be bound by theory, the activating compounds disclosed herein can activate TRPM8 and/or promote thermogenesis in one or more adipocytes. The activation of TRPM8 can promote thermogenesis or thermogenesis can be directly promoted after contact between the activating compound and one or more adipocytes. The activation of TRPM8 and/or the promotion of thermogenesis can lead to preferential formation of beige and brown adipocytes over white adipocytes from preadipocyte cells. Additionally, the activation of TRPM8 and/or the promotion of thermogenesis can lead to the conversion of white adipocytes to beige and/or brown adipocytes. Additionally, the activation of TRPM8 and/or the promotion of thermogenesis can lead to increased mitochondrial activity in white adipocytes, which may make them act more like beige or brown adipocytes.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

The foregoing summary is not intended to define every aspect of the invention, and additional aspects are described in other sections, such as the Detailed Description. In addition, the invention includes, as an additional aspect, all embodiments of the invention narrower in scope in any way than the variations defined by specific paragraphs set forth herein. For example, certain aspects of the invention that are described as a genus, and it should be understood that every member of a genus is, individually, an aspect of the invention. Also, aspects described as a genus or selecting a member of a genus should be understood to embrace combinations of two or more members of the genus. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

Features of the compositions and methods are described below. Section headings are for convenience of reading and not intended to be limiting per se. The entire document is intended to be related as a unified disclosure, and it should be understood that all combinations of features described herein are contemplated, even if the combination of features are not found together in the same sentence, or paragraph, or section of this document. It will be understood that any feature of the methods or compounds described herein can be deleted, combined with, or substituted for, in whole or part, any other feature described herein.

All measurements referred to herein are made at 25° C. unless otherwise specified.

As used herein, the word “or” when used as a connector of two or more elements is meant to include the elements individually and in combination; for example, X or Y, means X or Y or both.

The components of the present compositions are described in the following paragraphs.

The term “adipocyte”, as used herein, refers to a cell primarily composing adipose tissue, which specializes in storing energy as fat or triglycerides.

The term “white adipocyte”, as used herein, refers to an adipocyte whose main function is to act as a reservoir of triglycerides or fat for future energy utilization.

The term “brown adipocyte”, as used herein, refers to an adipocyte whose main function is to convert excess energy into body heat using non-shivering thermogenesis. Brown adipocytes are characterized by having a high proportion of mitochondria.

The term “beige adipocyte”, as used herein, refers to a white-like adipocyte that can induce non-shivering thermogenesis.

The term “lower”, as used herein in reference to a “lower alkyloxy” or a “lower alkylthio,” among others, refers to an alkyl chain of from 1 to 10 carbon atoms in length attached to the named functional group. For example, a “lower alkoxy” refers to an alkyl chain of 1 to 10 carbon atoms in length attached to a —OCH₃ functional group.

| SEQ ID NO | Sequence |
|---|---|
| 1 | Human TRPM8 DNA sequence |

A sequence listing that sets forth the nucleotide sequence for SEQ ID NO: 1 herein is being filed concurrently with the present application as an ASCII text file titled “15371_Nucleotide_Sequence_Listing_ST25.” The ASCII text file was created on 7 Nov. 2018 and is 5 Kbytes in size. In accordance with MPEP § 605.08 and 37 CFR § 1.52(e), the subject matter in the ASCII text file is incorporated herein by reference.

The term “TRPM8” or “TRPM8 receptor”, as used herein, refers to cold- and menthol-sensitive receptor (CMR1) or TRPM8. The TRPM8 nomenclature for the receptor comes from its characterization as a non-selective cation channel of the transient receptor potential (TRP) family that is activated by stimuli including low temperatures, menthol and other chemical coolants. The TRPM8 receptor is provided as SEQ ID NO: 1.

The cooling receptor conventionally known as TRPM8 or the menthol receptor has been demonstrated as a means to differentiate intensity and duration of organic molecules that initiate and propagate the non-thermal cooling perception (D. D. McKemy, The Open Drug Discovery Journal 2:81-88 2010). McKemy reported the EC50 values of many agonists to TRPM8 which span the range of 100 nM to 19 mM, thus showing the channel can be activated across a wide range of structures at varying concentrations. This channel also has the nomenclature of CRM1 and TRPP8. The later was designated as such due to its identification with prostate cells, where it was employed as a means to identify molecules targeted towards prostate cancer.

As stated previously, the present invention is directed to the discovery that specific 5-methyl-2-(1-methylethyl)-N-(2-phenylethyl)-, (1R, 2S, 5R) cyclohexanecarboxamide structures, as shown below, deliver the means to activate adipose tissue. Such activating compounds are described below.

Activating compounds are any such compounds or mixtures of compounds that can activate adipose tissue to induce thermogenesis. Examples of activating compounds include certain cyclohexanecarboxamide derivatives. Other examples of activating compounds that can be used to activate adipose tissue include compounds that can be described by Formula I. The activating compounds can also be salts of the compounds in Formula I.

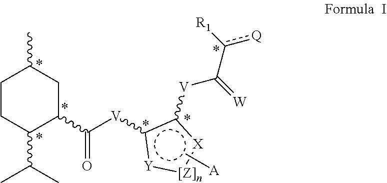

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and
stereochemistry is variable at the positions marked*.

Other activating compounds that can be used to activate adipose tissue can be described by Formula II. The activating compounds can also be salts of the compounds in Formula II.

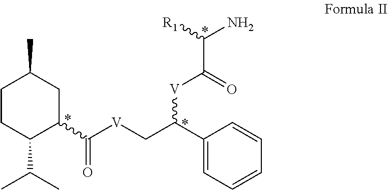

R₁ is H, alkyl, amino alkyl, or alkoxy;
V is —O— or —(NH)—; and
stereochemistry is variable at the positions marked*.

The activating compound can also be selected from the group consisting of the following formulae (Formulas III, IV, V, VI, and salts of Formulas III-VI).

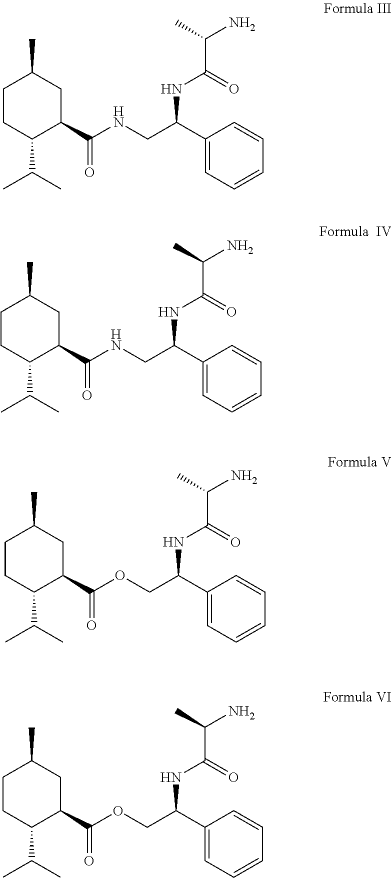 [SMILES: CC(C)[C@@H]1CC[C@@H](C)C[C@H]1C(=O)NC[C@@H](NC(=O)[C@H](C)N)c1ccccc1 ; CC(C)[C@@H]1CC[C@@H](C)C[C@H]1C(=O)NC[C@@H](NC(=O)[C@@H](C)N)c1ccccc1 ; CC(C)[C@@H]1CC[C@@H](C)C[C@H]1C(=O)OC[C@@H](NC(=O)[C@H](C)N)c1ccccc1 ; CC(C)[C@@H]1CC[C@@H](C)C[C@H]1C(=O)OC[C@@H](NC(=O)[C@@H](C)N)c1ccccc1]

Salts of Formula I-VI can include any acceptable salt of an activating compound represented by Formula I-VI. An acceptable salt is a salt that can be used in a formulation to be administered to humans. Suitable non-limiting examples of salts of Formula I-VI include Formula VII-IX.

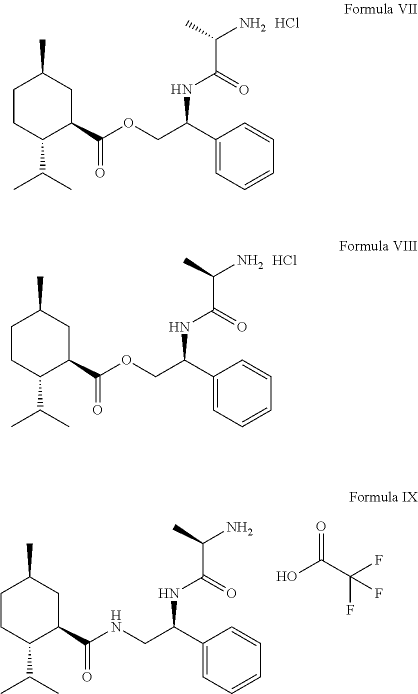 [SMILES: CC(C)[C@@H]1CC[C@@H](C)C[C@H]1C(=O)OC[C@@H](NC(=O)[C@H](C)N)c1ccccc1 ; Cl ; CC(C)[C@@H]1CC[C@@H](C)C[C@H]1C(=O)OC[C@@H](NC(=O)[C@@H](C)N)c1ccccc1 ; Cl ; CC(C)[C@@H]1CC[C@@H](C)C[C@H]1C(=O)NC[C@@H](NC(=O)[C@@H](C)N)c1ccccc1 ; O=C(O)C(F)(F)F]

The activating compound can be applied either as the sole active ingredient or in combination with other active ingredients. Some examples of other active ingredients include, but are not limited to, beta-3 adrenergic receptor agonists, such as mirabegron or solabegron.

The activating compound can also include metabolites and/or biologically accessible derivatives of the compounds from Formula I-IX.

The activating compound can be applied to an affected area. The affected area can be throughout the body, wherein the activating compound can enter the body through ingestion of a pill comprising the activating compound. The affected area can be a targeted location on the body or locations on the body. The affected area can be an area that has an excess of adipose tissue. The affected area can have an excess of adipose tissue from the perspective or opinion of a person in need of such treatment. The affected area can have an excess of adipose tissue from the perspective or opinion of a medical professional. The affected area can have an excess of white adipose tissue. The affected area can have an excess of adipose tissue for cosmetic or aesthetic purposes. Whether the affected area can have an excess of adipose tissue for cosmetic or aesthetic purposes can be determined by a person in need of such treatment, a medical professional, or a third-party observer.

Adipose tissue can be selected from the group consisting of brown adipocytes, white adipocytes, beige adipocytes, brite adipocytes, subcutaneous adipose tissue, pericardial adipose tissue, marrow adipose tissue, and/or combinations thereof. Excess adipose tissue can be found beneath the skin (i.e. subcutaneous fat), around internal organs (i.e. visceral fat), in bone marrow (i.e. yellow bone marrow), intermuscular (i.e. within the Muscular system) and in breast tissue. An affected area can include excess adipose tissue found in subcutaneous adipose tissue, visceral adipose tissue, yellow bone marrow, intermuscular adipose tissue, and/or breast tissue.

Persons in need of such treatment can include a person or animal that has an affected area with an excess of adipose tissue. Persons in need of such treatment can have an affected area, multiple affected areas, or have a disease that is commonly associated with excess adipose tissue, such as type 1 diabetes, type 2 diabetes, insulin-resistance, dyslipidemia, irritable bowel syndrome, chronic pain, neuropathic pain, and/or inflammatory pain. Additionally, persons in need of such treatment can also include a person or lower animal that uses the treatment for body contouring, body shaping and/or obesity. Body contouring and body shaping can be used as a treatment for a single affected area or multiple affected areas.

While not wishing to be bound by scientific theory, the method can further comprise the step of activating a receptor. After the activating compound is applied to the affected area, the receptor can be activated by the activating compound. The receptor can be TRPM8, alpha adrenergic receptors, beta adrenergic receptors, gamma adrenergic receptor, PPARGC1A, and/or combinations thereof.

While not wishing to be bound by scientific theory, the method can further comprise the step of expressing a mitochondrial protein. After activating compound is applied to the affected area, the mitochondrial protein can be expressed. The mitochondrial protein can be UCP1, UCP2, PPARGC1A, PRDM 16, ACADM, CPT1A, FASN, and/or combinations thereof. The mitochondrial protein can be found within white adipocytes, beige adipocytes, and/or brown adipocytes.

While not wishing to be bound by scientific theory, the method can further comprise the step of activating adipose tissue to induce thermogenesis. After activating compound is applied to the affected area, adipose tissue can be activated to induce non-shivering thermogenesis. The adipose tissue can be activated to induce diet-induced thermogenesis.

While not wishing to be bound by scientific theory, the method can further comprise the steps of activating a receptor, expressing a mitochondrial protein, and/or activating adipose tissue to induce thermogenesis.

One or more adipocytes can be contacted with the activating compound using any effective means. A means for contacting the one or more adipocytes with an activating compound is any means that allows for the activating compound to directly access the adipose tissue and/or one or more adipocytes. Some suitable routes of contact include, but are not limited to, injection, buccal, enteral, inhalable, infused, intramuscular, intrathecal, intravenous, nasal, ophthalmic, oral, otic, rectal, subcutaneous, sublingual, topical, transdermal, vaginal and/or combinations thereof.

One or more adipocytes can be contacted with the activating compound can be contacted in any form suitable for safely and effectively delivering the activating compound to the affected area. Some forms the activating compound can include, but are not limited to, tablet, pill, suppository, micro-needle patch, transdermal patch, suspension, solution, body wrap, and/or combinations thereof.

Disclosed herein is a device comprising a therapeutically effective amount of an activating compound and a means for contacting the activating compound with adipose tissue.

For administration to humans, or other mammalian subjects, especially pet animals, in need of such treatment, the total daily dose of the compounds of formula (I-VI) depends, on the mode of administration. For example, oral administration may require a higher total daily dose, than an intravenous dose. The total daily dose may be administered in single or divided doses. A therapeutically effective amount of the activating compound is an amount of activating compound that can induce the intended effect. Some intended effects include, but are not limited to, promotion of thermogenesis, activation of adipose tissue, adipocyte differentiation, the conversion of white adipocytes to beige and/or brown adipocytes, reduction in size and/or quantity of adipose tissue, body contouring, body shaping, and or the treatment of obesity, type 1 diabetes, type 2 diabetes, insulin resistance, dyslipidemia, irritable bowel syndrome, chronic pain, neuropathic pain, and/or inflammatory pain.

A therapeutically effective amount means an amount of the activating compound or composition comprising the activating compound sufficient to induce a positive benefit, a health benefit, and/or an amount low enough to avoid serious side effects, i.e., to provide a reasonable benefit to risk ratio, within the sound judgment of a skilled artisan. A therapeutically effective amount can mean at least 0.01% of the activating compound, by weight of the composition, alternatively at least 0.1%. A therapeutically effective amount can be determined as the mass of the activating compound per kg of body weight of the individual. A therapeutically effective amount can mean at least 0.0001 mg/kg of body weight.

One or more adipocytes can be contacted with an activating compound in a treatment regimen. In a treatment regimen, the activating compound can be administered in a predetermined schedule. For example, an activating compound can be administered daily, weekly, monthly, and/or quarterly. Additionally, an activating compound can be administered in single and/or multiple doses.

The device can comprise a means for contacting the activating compound with adipose tissue. Suitable means for contacting the activating compound with adipose tissue include any equipment needed to apply the activating compound to the affected area. For example, injection would be a suitable means for contacting an activating compound in a syringe with subcutaneous adipose tissue. Some examples of means for contacting the activating compound with adipose tissue include, but are not limited to injection, buccal, enteral, inhalable, infused, intramuscular, intrathecal, intravenous, nasal, ophthalmic, oral, otic, rectal, subcutaneous, sublingual, topical, transdermal, and/or combinations thereof. Oral administration can be accomplished with a pill, tablet, solution, suspension, slurry, and/or other common formulations for orally ingesting an active ingredient. Transdermal administration can be accomplished with a micro-needle patch, transdermal patch, fabric wrap, paper, seaweed wrap, and combinations thereof.

Disclosed herein is an activating compound for use as a medicament. The activating compound can be chosen from any one of the compounds represented by Formulas I-VI. Disclosed herein is an activating compound for use in the treatment of obesity. Disclosed herein is an activating compound for use in the treatment of type 1 diabetes, type 2 diabetes, insulin-resistance, dyslipidemia, irritable bowel syndrome, chronic pain, neuropathic pain, and/or inflammatory pain. Use of an activating compound for the manufacture of a medicament for the treatment of obesity. Disclosed herein is the use of an activating compound for the manufacture of a medicament for the treatment of obesity, type 1 diabetes, type 2 diabetes, insulin-resistance, dyslipidemia, irritable bowel syndrome, and/or chronic pain, neuropathic pain, and/or inflammatory pain. Disclosed herein is an activating compound for use in body contouring. Disclosed herein is an activating compound for use in body shaping. Disclosed herein is an activating compound for use in the reduction of the size and/or quantity of adipose tissue; use of an activating compound for the manufacture of a medicament for the treatment of body contouring; use of an activating compound for the manufacture of a medicament for the treatment of body shaping; and use of an activating compound for the manufacture of a medicament for the treatment of the reduction of the size and/or quantity of adipose tissue.

Disclosed herein are stereoisomerically pure activating compounds. A stereoisomerically pure activating compound is an activating compound that does not contain mixtures of stereoisomers, i.e. compounds with the same molecular formula, but with different chirality at one or multiple locations on the molecule. Disclosed herein are enantiomerically pure activating compounds. An enantiomerically pure activating compound is an activating compound that does not contain mixtures of enantiomers, i.e. stereoisomers that are mirror images of each other that are non-superimposable. Any method for isolating enantiomerically pure compounds of Formula I-VI can be used, such as, for example, as set forth in U.S. Pub. App. No. 2017/0036994, which is herein incorporated by reference.

The present invention is also directed to lotion compositions. A lotion composition of the present invention comprises at least one rheology structurant, which typically is a solid. The lotion composition can further comprise other optional ingredients, like surface energy modifiers. In one embodiment, a lotion composition consists essentially of, or consists of, a rheology structurant, such as a microcrystalline wax, alkyl dimethicone, ethylene glycol dibehenate, ethylene glycol distearate, glycerol tribehenate, glycerol tristearate, and ethylene bisoleamide. A present lotion composition can contain a single rheology structurant or a mixture of two or more rheology structurants.

In preparing a lotioned catamenial device according to the present invention, the lotion composition can be applied to the outer surface of the absorbent article, such as, for example, the outer surface of the topsheet. Any of a variety of application methods that distribute lubricious materials having a molten or liquid consistency can be used, such as, for example, as set forth in U.S. Pat. No. 5,968,025 and U.S. Pub. App. No. 2005/0208113. Suitable methods include but are not limited to spraying, printing (e.g., flexographic printing), coating (e.g., gravure coating), extrusion, dipping, or combinations of these application techniques, e.g., spraying the lotion composition on a rotating surface, such as a calender roll, that then transfers the composition to the outer surface of the sanitary napkin topsheet. Additionally, the manner of applying the lotion composition to a portion of a catamenial device can be such that the substrate or component does not become saturated with the lotion composition. The lotion composition can be applied to the catamenial device at any point during assembly. For example, the lotion composition can also be applied to the outer surface of the topsheet before it is combined with the other raw materials to form a finished catamenial device.

### Method of Promoting Thermogenesis

A. A method of promoting thermogenesis comprising contacting one or more adipocytes with an activating compound with one or more adipocytes, wherein the activating compound comprises the following structure or salts thereof:

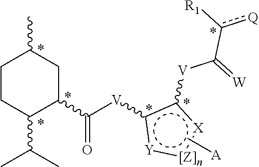

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and
stereochemistry is variable at the positions marked*.
   B. The method according to paragraph A, wherein the activating compound comprises the following structure or salts thereof:

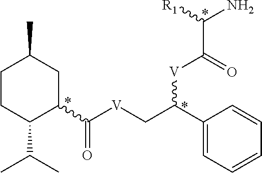

R₁ is H, alkyl, amino alkyl, or alkoxy;
V is —O— or —(NH)—; and
stereochemistry is variable at the positions marked*.
   C. The method according to paragraph A or B, wherein the activating compound is selected from the group consisting of:

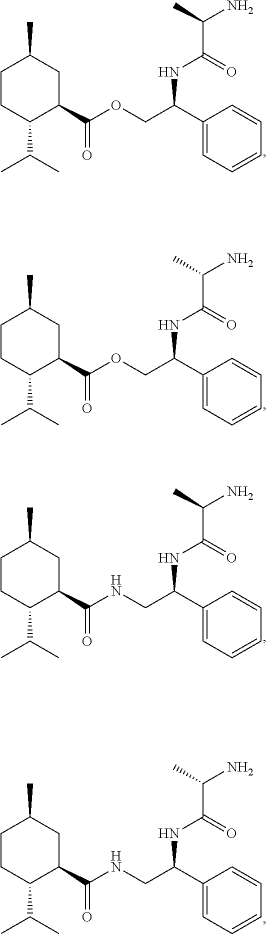 [SMILES: CC(C)[C@@H]1CC[C@@H](C)C[C@H]1C(=O)OC[C@@H](NC(=O)[C@@H](C)N)c1ccccc1 ; CC(C)[C@@H]1CC[C@@H](C)C[C@H]1C(=O)OC[C@@H](NC(=O)[C@H](C)N)c1ccccc1 ; CC(C)[C@@H]1CC[C@@H](C)C[C@H]1C(=O)NC[C@@H](NC(=O)[C@@H](C)N)c1ccccc1 ; CC(C)[C@@H]1CC[C@@H](C)C[C@H]1C(=O)NC[C@@H](NC(=O)[C@H](C)N)c1ccccc1]

and salts thereof.
D. The method according to any one of paragraphs A-C, wherein the method further comprises the steps of:
expressing a mitochondrial protein; and
activating one or more adipocytes to induce thermogenesis.
   E. The method according to any one of paragraphs A-D, wherein the mitochondrial protein is selected from the group consisting of Ucp1, Ucp2, and combinations thereof.
   F. The method according to any one of paragraphs A-E, wherein the method further comprises activating a receptor upon contact of activating compound with one or more adipocytes.
   G. The method according to any one of paragraphs A-F, wherein the receptor is selected from the group consisting of TrpM8, PPARGC1A, alpha adrenergic receptor, beta adrenergic receptor, and gamma adrenergic receptor.
   H. The method according to any one of paragraphs A-G, wherein one or more adipocytes are present in an affected area.
   I. The method according to any one of paragraphs A-H, wherein the affected area has an excess of adipose tissue.
   J. The method according to any one of paragraphs A-I, wherein the adipose tissue is selected from the group consisting of brown adipocytes, white adipocytes, beige adipocytes, brite adipocytes, subcutaneous adipose tissue, pericardial adipose tissue, marrow adipose tissue, and combinations thereof.
   K. The method according to any one of paragraphs A-J, wherein the treatment reduces the size and quantity of white adipocytes.
   L. The method according to any one of paragraphs A-K, wherein an individual is treated by contacting the activating compound with one or more adipocytes.
   M. The method according to any one of paragraphs A-L, wherein the treatment is selected from the group consisting of the treatment of obesity, the reduction of adipose tissue, body contouring, body shaping, type 1 diabetes, type 2 diabetes, insulin-resistance, dyslipidemia, irritable bowel syndrome, chronic pain, neuropathic pain, and inflammatory pain.
   N. The method according to any one of paragraphs A-M, wherein the activating compound is contacted with one or more adipocytes through a route selected from the group consisting of injection, buccal, enteral, inhalable, infused, intramuscular, intrathecal, intravenous, nasal, ophthalmic, oral, otic, rectal, subcutaneous, sublingual, topical, transdermal, and combinations thereof.
   O. The method according to any one of paragraphs A-N, wherein the activating compound is contacted with one or more adipocytes in a form selected from the group consisting of tablet, pill, suppository, micro-needle patch, transdermal patch, suspension, solution, body wrap, and combinations thereof.

### Method of Treatment

A. A method of treatment comprising contacting one or more adipocytes with an activating compound, wherein the activating compound comprises the following structure or salts thereof:

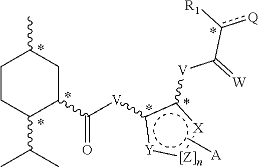

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and
stereochemistry is variable at the positions marked*.
   B. The method according to paragraph A, wherein the activating compound comprises the following structure or salts thereof:

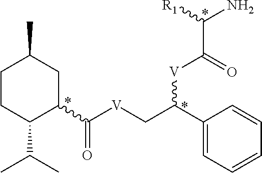

R₁ is H, alkyl, amino alkyl, or alkoxy;
V is —O— or —(NH)—; and
stereochemistry is variable at the positions marked*.
   C. The method according to paragraph A or B, wherein the activating compound is selected from the group consisting of:

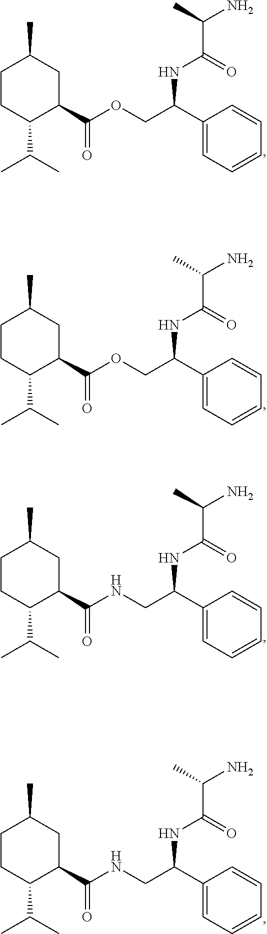 [SMILES: CC(C)[C@@H]1CC[C@@H](C)C[C@H]1C(=O)OC[C@@H](NC(=O)[C@@H](C)N)c1ccccc1 ; CC(C)[C@@H]1CC[C@@H](C)C[C@H]1C(=O)OC[C@@H](NC(=O)[C@H](C)N)c1ccccc1 ; CC(C)[C@@H]1CC[C@@H](C)C[C@H]1C(=O)NC[C@@H](NC(=O)[C@@H](C)N)c1ccccc1 ; CC(C)[C@@H]1CC[C@@H](C)C[C@H]1C(=O)NC[C@@H](NC(=O)[C@H](C)N)c1ccccc1]

and salts thereof.
D. The method according to any one of paragraphs A-C, wherein the method further comprises the steps of:
expressing a mitochondrial protein; and
activating one or more adipocytes to induce thermogenesis.
   E. The method according to any one of paragraphs A-D, wherein the mitochondrial protein is selected from the group consisting of Ucp1, Ucp2, and combinations thereof.
   F. The method according to any one of paragraphs A-E, wherein the method further comprises activating a receptor upon contact of activating compound with one or more adipocytes.
   G. The method according to any one of paragraphs A-F, wherein the receptor is selected from the group consisting of TrpM8, PPARGC1A, alpha adrenergic receptor, beta adrenergic receptor, and gamma adrenergic receptor.
   H. The method according to any one of paragraphs A-G, wherein one or more adipocytes are present in an affected area.
   I. The method according to any one of paragraphs A-H, wherein the affected area has an excess of adipose tissue.
   J. The method according to any one of paragraphs A-I, wherein the adipose tissue is selected from the group consisting of brown adipocytes, white adipocytes, beige adipocytes, brite adipocytes, subcutaneous adipose tissue, pericardial adipose tissue, marrow adipose tissue, and combinations thereof.
   K. The method according to any one of paragraphs A-J, wherein the treatment reduces the size and quantity of white adipocytes.
   L. The method according to any one of paragraphs A-K, wherein an individual is treated by contacting the activating compound with one or more adipocytes.
   M. The method according to any one of paragraphs A-L, wherein the treatment is selected from the group consisting of the treatment of obesity, the reduction of adipose tissue, body contouring, body shaping, type 1 diabetes, type 2 diabetes, insulin-resistance, dyslipidemia, irritable bowel syndrome, chronic pain, neuropathic pain, and inflammatory pain.
   N. The method according to any one of paragraphs A-M, wherein the activating compound is contacted with one or more adipocytes through a route selected from the group consisting of injection, buccal, enteral, inhalable, infused, intramuscular, intrathecal, intravenous, nasal, ophthalmic, oral, otic, rectal, subcutaneous, sublingual, topical, transdermal, and combinations thereof.
   O. The method according to any one of paragraphs A-N, wherein the activating compound is contacted with one or more adipocytes in a form selected from the group consisting of tablet, pill, suppository, micro-needle patch, transdermal patch, suspension, solution, body wrap, and combinations thereof.

### Device

A. A device comprising
a therapeutically effective amount of an activating compound and
a means for contacting the one or more adipocytes with the activating compound.
   B. The device of paragraph A, wherein the activating compound comprises the following structure:

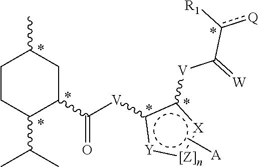

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and stereochemistry is variable at the positions marked*.
   C. The device of paragraph A or B, wherein the activating compound comprises the following structure or salts thereof:

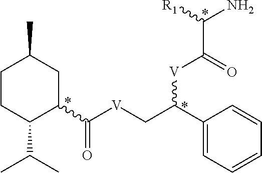

R₁ is H, alkyl, amino alkyl, or alkoxy;
V is —O— or —(NH)—; and
stereochemistry is variable at the positions marked*.
   D. The device of any one of paragraphs A-C, wherein the means for contacting the activating compound with one or more adipocytes is selected from the group consisting of injection, buccal, enteral, inhalable, infused, intramuscular, intrathecal, intravenous, nasal, ophthalmic, oral, otic, rectal, subcutaneous, sublingual, topical, transdermal, and combinations thereof.

### Medicament

A. Formula I-VI for use as a medicament.

### Treatment of Obesity

A. Formula I-VI for use in the treatment of excess adipose tissue.

### EXAMPLES

All EXAMPLES were run at room temperature (RT, 20° C.), standard pressure and atmosphere, unless otherwise noted. The water used in the EXAMPLES was deionized water, unless otherwise noted.

### TRPM8 Protocol-FLIPR Assay

To determine whether TRPM8 is activated, the intracellular calcium ion (Ca²⁺) level was measured from transfected cells with the TRPM8 receptor sequence (SEQ ID NO: 1). HEK-293 (human embryonic kidney) cells stably transfected with human TRPM8 were grown in 15 mL growth medium (high glucose DMEM (Dulbecco's Modification of Eagle's Medium) supplemented with 10% FBS (fetal bovine serum), 100 μg/mL penicillin/streptomycin, 5 μg/mL blasticindin, and 100 μg/mL zeocin) in a 75 cm² flask for 3 days at 37° C. in a mammalian cell culture incubator (Forma Scientific Model 3110, Marietta, Ohio) set at 5% CO₂. Cells were detached with addition of 2 mL of trypsin-EDTA buffer (GIBCO® 25200, Invitrogen, Grand Island, N.Y.) for about 2-3 min. Trypsin was inactivated by addition of 8 mL growth medium. Cells were transferred to a 50 mL tube and centrifuged at 850 rpm for 3 minutes to remove medium. After centrifugation, a pellet of cells was formed in the bottom of the tube separating them from the supernatant solution. The supernatant was discarded and the cell pellet was suspended in 1 mL of fresh growth medium to which 5 μL (12.5 μg) of Fluo-4 AM (Molecular Probes, Inc., Eugene, Oreg.) calcium indicator was added and incubated for 30 min with gentle shaking. Fluo-4 AM is a fluorescent dye used for quantifying cellular Ca²⁺ concentrations in the 100 nM to 1 μM range. At the end of 30 minutes, 45 mL of assay buffer (1×HBSS (Hank's Balanced Salt Solution), 20 mM HEPES (4-(2-Hydroxyethyl)-1-piperazineethanesulfonic acid)) was added to wash cells and the resulting mixture was then centrifuged at 850 rpm for 3 minutes at 20° C. to remove excess buffer and Fluo-4 AM calcium indicator.

The pelleted cells were re-suspended in 10 mL assay buffer and 90 μL aliquots (˜50,000 cells) per well delivered to a 96-well assay plate containing 10 μL of test compounds (1 mM in assay buffer, final concentration 100 μM) or buffer control and incubated at room temperature for 30 minutes. After 30 minutes, a plate (Falcon 353219, Corning Corning N.Y.) was placed into a fluorometric imaging plate reader (FLIPR384 from Molecular Devices, Sunnyvale, Calif.) and basal fluorescence recorded (excitation wave length 488 nm and emission wave length 510 nm). Then 20 μL of 100 mM of TRPM8 agonist WS5 coolant in the assay buffer was added and fluorescence recorded. For determining the direct effect of test compounds on TRPM8, fluorescence was measured immediately after addition of each compound (TABLE 1). Additional discussion of the FLIPR method can be found in Smart et al., Characterization using FLIPR of human vanilloid VR1 receptor pharmacology, European Journal of Pharmacology 417, 51-58 (2001) and Liu et al., Development and validation of a platelet calcium flux assay using a fluorescent imaging plate reader, Analytical Biochemistry 357, 216-224 (2006).

The magnitude of the fluorescence of the active-treated cells was compared to the magnitude of the fluorescence from a benchmark agonist (WS-5), as described above. The percentage of fluorescence as a function of active dose was plotted and a sigmoidal curve was generated. Curve fitting from this dose-response curve yielded the value for TRPM8 IC₅₀ in nM.

### Lipogenesis Assay

Lipogenesis is the process by which acetyl-CoA is converted to fatty acid. Through lipogenesis and subsequent triglyceride synthesis, energy can be stored in the form of adipose tissue (i.e. fats). To determine what effect, if any, activating compounds (shown in TABLE 1) had on lipogenesis, the protocol listed below was used.

To determine whether the activating compounds (shown in TABLE 1) impacted the formation of white adipocytes from pre-adipocytes, a lipogenesis assay was performed. The cells utilized in the protocol were cryo-preserved Human subcutaneous pre-adipocytes superlot (Zen-Bio, Inc., Research Triangle Park, N.C., Cat # SP-F-SL). The growth media used was PM-1 (Zen-Bio, Inc., Research Triangle Park, N.C., cat # PM-1) plus 5 ng/mL Epidermal Growth Factor (EGF). The PM-1+5 ng/mL EGF was prepared by adding 12.5 uL of 200 ug/mL EGF stock to 500 mL PM-1. The differentiation media used was DM-2 (Zen-Bio, Inc., Research Triangle Park, N.C., cat # DM-2). The adipocyte maintenance media used was AM-1 (Zen-Bio, Inc., Research Triangle Park, N.C., cat # AM-1).

First, the human subcutaneous pre-adipocyte cells were thawed in a 37° C. water bath. Next, the thawed pre-adipocyte cells were added to 9 mL of growth medium (PM-1) at 20° C. in a 15 mL polypropylene tube (Bioexpress, Corning, Corning, N.Y.). The tube was centrifuged at 280×g (˜1100 rpm) at 20° C. for 5 min. After 5 min, the supernatant was removed and the accumulated solids were re-suspended in 2 mL of PM-1 using trituration. The suspension of cells was counted under 100× magnification using a Cyto C-Chip hemacytometer (Incyto, Seoul, South Korea, cat # DHC-N01-5). In order to proceed, 15-50 cells per 1×1 mm square in hemacytometer was needed. If, the number of cells per square was not met, 6.7×10⁵ (670,000) cells per T75 flask (Bioexpress, Corning, Corning, N.Y.) were added to each square and the suspension was diluted to 20 mL with PM-1. Every other day, new media was added to replace media removed due to metabolism by the cells. The cells were re-counted as described previously. Cells were grown to 80-90% confluence, which took approximately 4-5 days. 80-90% confluence is determined by visual inspection of the area of the flask occupied by the cells. When 80-90% of the flask area has been occupied, cells are at 80-90% confluence. Cell colonies were then split 1:6 by harvesting from a single flask and equally dividing the harvested cells into six flasks. Colonies were not allowed to exceed 3 passages and were not grown to complete confluence. The pre-adipocytes were grown until they contact with other cells in the flask to aid differentiation into white or brown adipocytes.

After the pre-adipocytes were sufficiently grown to 90% confluence, the cells were ready for differentiation and treatment with an activating compound. Cells were washed with 3 mL of PBS and detached using 3 mL of Trypsin EDTA. The cells were incubated at 37° C. for 5 minutes. Next, the cells were centrifuged at 280×g at 20° C. for 5 minutes. The supernatant was discarded and the resulting pellet was re-suspended in 10 mL of PM-1 using trituration. Cells were counted at 100× magnification and the cells were diluted to 86,667 cells/mL (˜13,000 cells/150 μL) by adding the necessary amount of PM-1 to achieve the desired concentration. Upon addition of PM-1, samples were swirled by hand to evenly disperse the cells prior to plating. Once at the desired concentration (86,667 cells/mL, cells were plated at 13,000 cells (i.e. using 150 μL of suspension) in 96-well plate (#3595, Corning, Corning, N.Y.). Cells were cultured between 24-48 hours to confluence in CO₂-incubator at 37° C. If cells did not achieve confluence by 48 hours, samples were not used.

Once confluence is reached, 150 μL of differentiation media (DM-2) was added (DAY 0). Samples were incubated at 37° C. for 6 days. On DAY 6, 90 μL of media was removed from each sample via aspiration, without touching the bottom of the well. Next, 140 μL of adipocyte maintenance medium (AM-1) was added. The AM-1 ran down the side of well. On DAY 6, activating compounds were added (2 μL of 10 mM activating compound) to give a final concentration of 100 μM of activating compounds. All samples were incubated at 37° C. for 9 more days without changing the media or shaking the samples.

The positive control to the activating compounds was Genistein (Sigma-Aldrich, St. Louis, Mo., Sigma cat # G6649). A 5 mM stock solution of Genistein (5 mg) in dimethyl sulfoxide (DMSO, 3.7004 mL) was prepared. 2 μL of Genistein stock solution was added to each positive control well to give a final concentration of 50 μM. All samples were incubated at 37° C. for 9 more days without changing the media.

Next, the samples were stained to aid in lipogenesis quantitation. 5 μL of AdipoRed (Lonza Group, Basel, Switzerland, cat # PT07009) was added directly to the cells in the 96-well cell treatment plate. Each row was mixed gently by tapping the plate on the side of lab bench. Samples were incubated for at least 15 min at room temperature (˜20° C.). Lipogenesis was quantified using an Envision Fluorescent spectrophotometer Plate Reader (PerkinElmer, Waltham, Mass., cat #3595). The “Copy of AdipoRed” protocol was utilized on the software provided with the Envision spectrophotometer. The plates were scanned from the bottom using the 451 mirror (excitation 485 nm; emission 535 nm). Each well was scanned in a Z pattern because, as the cells acquire triglycerides, some can float off, especially toward the middle of the well (7 reads across from left to right, 7 reads diagonally from right to left and 7 reads across from left to right for a total of 21 end points).

After the samples were scanned they were normalized to the solvent using FluoReporter® Blue Fluorometric dsDNA Quantitation Kit (Invitrogen, Carlsbad, Calif., cat # F2962). Immediately after the initial measurements, AdipoRed containing cell media was gently aspirated by tilting the vessel so that the aspiration pipette does not damage any cells. Cells were rinsed with 100 μL/well with PBS buffer. Special care was taken to not dislodge the samples from the bottom. Next, 100 μL of distilled water was added to each well. The plates were frozen at −80° C. to lyse the cells.

Plates were thawed later by removing them from the −80° C. freezer and allowing them to ambiently warm to room temperature (˜20° C.). 25 μL of Hoechst 33258 solution (Invitrogen, Carlsbad, Calif., cat # F2962, Component A) was added to 10.0 mL of TNE Buffer (Invitrogen, Carlsbad, Calif., cat # F2962, Component B). With large numbers of cells (>100,000), improved analytical linearity may be obtained by increasing the final concentration of Hoechst 33258 to 50 μL in 10.0 mL of TNE Buffer. 100 μL of aqueous Hoechst 33258 in TNE Buffer was then added to each well. Blank fluorescence wells were included, using 100 uL of aqueous Hoechst 33258 in TNE Buffer+100 uL ddH2O/well. Fluorescence was then measured using excitation at 360 nm and emission at 460 nm. The blank fluorescence values were subtracted from the sample test data. The normalization factor was then calculated using Equation I, below:

$$Normalization factor = \frac{Test Compound {RFU}^{1}}{Control {RFU}^{1}} \times 100 \% \\ {RFU}^{1} = average RFU of replicate walls from FluoReporter® Blue Flurometric dsDNA Quantitation . \\ Control = the control , DMSO or water , that matches the solvent used for the test compound . \qquad Equation I$$

Normalized AdipoRed value was calculated by dividing AdipoRed data by the normalization factor determined using Equation I. The normalized % Inhibition was calculated using Equation II.

$$Normalized \% Inhibition = \frac{( Control {RFU}^{2} - Test Compound {RFU}^{2} )}{Control {RFU}^{2}} \\ {RFU}^{2} = normalized average RFU of replicate wells from AdipoRed staining . \\ Control = the control , DMSO or water , that matches the solvent used for the test compound . \qquad Equation II$$

The % inhibition values were plotted out against the doses of treatment which resulted in a sigmoidal curve when inhibition was present. The curve was fit using Graphpad Prism software to calculate the Lipogenesis IC₅₀.

### Real Time PCR Analysis—In Vitro

For Real Time PCR analysis of adipocytes, cells from cultures grown in 24 well plates were collected. RNA was isolated using RNeasy Kit (Cat 74104, Qiagen, Germantown, Md.) using manufacturer's protocol. RNA was quantitated using the Nanodrop 1000 (Thermofisher, Waltham, Mass.). cDNA formation was carried out using PowerUp SYBR™ Green Master Mix and real time PCR carried out on QuantStudio 6 machine (ThermoFisher, Waltham, Mass.) per manufacturer protocol. ΔΔCT analysis was done using the Expression Suite software (ThermoFisher) purchased with the instrument.

### TRPM8 Activation—In Vitro

TRPM8 activation was determined by measuring intracellular calcium ion (Ca²⁺) level from transfected cells with the TRPM8 receptor gene, as described in EXAMPLE 1, the results of which are shown in TABLE 1. IC₅₀ values are provided in column 3 of TABLE 1, which measured the concentration of activating compound needed to reduce intracellular [Ca²⁺] by 50%. A lower intracellular [Ca²⁺] indicated TRPM8 was activated.

In fact, as shown in TABLE 1, Formula III-VI as well as Comparative Examples 1 and 2 had lower concentrations to reach the IC₅₀ for TRPM8 activation than WS-5. This indicated that Formula III-VI and Comparative Examples 1 and 2 were effective at activating TRMP8. The comparative examples only differed from Formula III and IV in enantiomeric purity. Comparative Examples 1 and 2 contained mixtures of the S and R enantiomers of Formula III and IV. Formula III reached IC₅₀ at 2 nM. Formula IV reached IC₅₀ at 8-10 nM. Formula V reached IC₅₀ at 340 nM. Formula VI reached IC₅₀ at 8 nM. Comparative Examples 1 and 2 also activated TRPM8 at concentrations of 8-10 and 10-12 nM respectively. In comparison, WS-5 led to an IC₅₀ value at a concentration of 2000 nM.

Lipogenesis inhibition was determined by measuring the fluorescence of samples treated with activating compounds after staining with AdipoRed, which enabled the quantification of intracellular lipid droplets. Lipid droplets are found within white adipocytes. IC₅₀ value represented the concentration of activating compound needed to reduce lipogenesis by 50%. Thus, the lipogenesis IC₅₀ measured the conversion of pre-adipocytes into white adipocytes. The values for lipogenesis IC₅₀ are found in column 2 of TABLE 1.

Comparative Example 1 reached a lipogenesis IC₅₀ at 800 PM. Comparative Example 2 reached a lipogenesis IC₅₀ at 800 μM. WS-5 reached an IC₅₀ at 500 μM. Menthol showed no inhibition of lipogenesis. Thus, while Comparative Example 1, Comparative Example 2, WS-5, and Menthol could activate TRPM8 as shown in column 3, TABLE 1, each required a higher concentration to inhibit lipogenesis.

Surprisingly, in comparison, Formula III, V, and VI each inhibited lipogenesis at low concentrations. For example, Formula III reached a lipogenesis IC₅₀ at 25 μM. Formula V reached a lipogenesis IC₅₀ at 80 μM. Formula VI reached a lipogenesis IC₅₀ at 40 PM. Thus, Formula III, V, and VI inhibited the conversion of pre-adipocytes into white adipocytes.

TABLE 2 shows the relative expression of mRNA in adipocytes treated with activating compounds. TABLE 2 used Real Time PCR to determine which proteins were expressed in adipocytes upon treatment with activating compounds. As described previously in TABLE 1, activating compounds resulted in an inhibition of the conversion of pre-adipocytes into white adipocytes. Real Time PCR values were relative to a control sample that was not treated with any activating compounds. Thus, values over 1 indicated that the mRNA of a particular protein was expressed more frequently in a treated sample. In WS-5 and Menthol, no Real Time PCR value was over 2.0, which indicated only slight changes in mRNA expression.

In samples treated with Formula IV, none of the PCR values were over 2. However, samples treated with Formula III displayed a UCP-1 PCR value of 5.8. Such a high PCR value indicated that there was a dramatic increase in UCP-1 mRNA expression. Beige and brown adipocytes have a high proportion of UCP-1 proteins. Thus, pre-adipocytes treated with Formula III displayed a decrease in lipogenesis (i.e. smaller rate of white adipocyte formation) and an increase in UCP-1 mRNA expression (i.e. higher rate of brown/beige adipocyte formation).

TABLE 1
Addition of Activating Compounds to Pre-Adipocyte Cells

| | Lipogenesis | TrpM8 |
|---|---|---|
| | IC₅₀ | IC₅₀ |
| Activating Compounds | (μM) | (nM) |
| Formula III | 25 | 2 |
| Formula IV | 800 | 8-10 |
| Formula V | 80 | 340 |
| Formula VI | 40 | 8 |
| 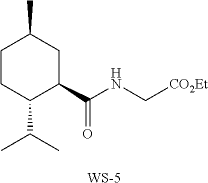 | 600 | 2000 |
| Menthol | NI | 7000 |
| 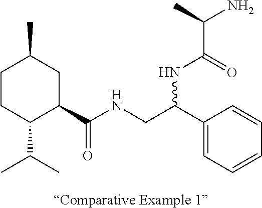 | 800 | 8-10 |
| 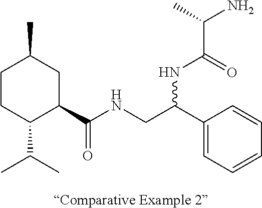 | 900 | 10-12 |

NI denotes that no inhibition was observed at concentration less than/equal to 1 mM

TABLE 2
Real Time PCR of Adipocytes after
Treatment with Activating Compounds

| | Relative Expression of mRNA in Adipocytes | | | | |
|---|---|---|---|---|---|
| | treated with Activating Compounds | | | | |
| Protein | Control | Formula III | Formula IV | WS-5 | Menthol |
| UCP-1 | 1 | 5.8* | 1.8 | 1.5 | 1.7 |
| PPARGC1A | 1 | 0.8 | 1.1 | 1.7 | 2.0 |
| PRDM16 | 1 | 0.6 | 0.7 | 1.3 | 0.75 |
| ACADM | 1 | 1.75 | 1.4 | 1.45 | 1.45 |
| CPT1A | 1 | 1.7 | 1.7 | 1.5 | 1.35 |
| FASN | 1 | 1.85 | 1.35 | 2.0 | 1.45 |
| 18S | 1 | 1 | 0.95 | 0.85 | 1 |
| GAPDH | 1 | 1 | 1 | 1 | 1 |

*p-value < 0.05

### In Vivo Browning Studies in Lean Mice—In Vivo

Mice (8-10 weeks old males C57BJ/6 strain) were obtained from a commercial vendor Charles River Laboratories. The animal study protocols were approved by the Institutional Animal Care and Use Committee, at the Procter & Gamble Company. Mice were acclimated to the facility for 14 days prior to initiating the study. From arrival, mice were housed in solid-bottom shoebox styled cages within room temperature of 22±2° C. with ad libitum access to water and regular rodent chow diet on a 12 h light/dark cycle. Mice were single housed and offered bedding and various enrichment options. The bedding and nesting material allowed the mice to thermoregulate to their desired level of comfort. Body weights were recorded at the beginning of the study, on each dosing day prior to dosing, and final weights were recorded at the end of the study. Overall food consumption, fecal output, and body appearance were monitored during cage side clinical observations but were not scientifically measured or tracked. During the dosing period, the animals were observed several times per day (for example, before and during injection, immediately to 30-min post injection, a few hours after injection, and end of work day).

Three groups of animals were treated with mirabegron (i.e. positive control) (n=4), placebo (n=2) and Formula III (n=6) on the left side while the right side received placebo in all three groups. The injection was performed using a 25-27-gauge ½-⅝″ length needle via a subcutaneous (SC) injection near or into the inguinal fat pad region of the lower abdomen. Dosing was done twice a week injections over 3 weeks. Necropsy was done at week 4. Test materials were administered at room temperature and in a neutral pH range. Three days after last dosing animals were euthanized with CO2 inhalation, inguinal fat pads were harvested, and tissues were processed for histology and biomarker analysis.

Histology was performed after H&E staining by Vet Path Services. Mice were euthanized by CO₂ asphyxiation, and the tissue samples were fixed in 10% formalin a minimum of 18 hours and then embedded in paraffin, cut into 5 m sections, and stained using hematoxylin and eosin (H&E) for histological analysis. The dosing scheme for the injections is provided in TABLE 3.

TABLE 3
Dosing Scheme per Injection

| | injection | | total dose/ |
|---|---|---|---|
| | volume | concentration | injection |
| | (uL) | (ug/mL) | (mg) |
| High Dose Formula III | 100 | 0.003 | 0.3 |
| Medium Dose Formula III | 100 | 0.00125 | 0.125 |
| Low Dose Formula III | 100 | 0.0006 | 0.06 |
| Medium Dose Mirabegron | 100 | 0.000125 | 0.0125 |
| Low Dose Mirabegron | 100 | 0.00006 | 0.006 |

Miragrebon was purchased from Selleck Chemicals S4009 (VWR 103543-358). All chemicals were purchased from Millipore-Sigma otherwise specified. The deionized water was prepared by a Millipore NanoPure purification system (resistivity higher than 18.2 MΩ2 cm-1) for buffer preparation. All materials were dissolved in PBS.

The confirmation study employed three mice. Formula III was injected at one side of the back leg and mirabegron at the other side, and one mouse got a high dosage, and the other two got the middle-level dosage. A biopsy sample was collected from each treatment site, and three history images from each biopsy sample were prepared to confirm the difference between treatment and control by the image analysis mentioned below.

A sliced tissue was prepared from each biopsy sample, both Formula III, and control treatment. H&E is used to stain the inter-cellular region to make an apparent contrast against the adipocyte region. Three images were captured from each sliced tissue by TIFF format.

Segmentation of Adipocyte from an Image

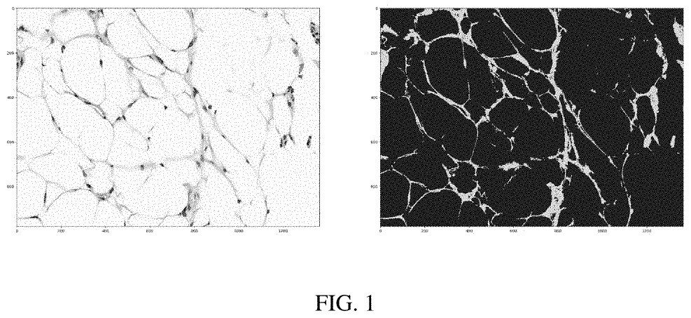

Segments of adipocyte and inter-cellular regions in a stained image were identified by Python scikit-learn package. All the color images were first converted to grayscale with the cv2 function of the OpenCV-python library. The threshold between adipocyte and inter-cellular regions in a converted image was detected by the Otsu filter function of the scikit-learn. The detected value was adjusted by multiplying 1.1. FIG. 1 shows an example of segmentation (right) from a stained image (left).

The ratio of adipocyte in an image was calculated from the pixel number of the segments—Adipocyte Pixel was divided by Total Pixel. TABLE 4 shows the Fat % compilation of the detected segments from each image of the treatment and control subjects.

TABLE 4
Fat %

| | Average Fat % | Standard Deviation | p-value |
|---|---|---|---|
| Formula III | 90.81% | 4.11% | 0.0278 |
| Mirabegron | 94.43% | 3.54% | — |

The significance of the difference in Adipocyte Ratio between treatment and control groups was assessed by the Negative Binomial Generalized Linear Mixed-Effects Model using glmer.nb function of R lme4 package. Adipocyte Pixel was modeled with Treatment Group as a fixed factor, subject as a random factor, and Total Pixel as an offset value. The high and midlevel-level dosage subjects were merged as a treatment group in this analysis. The pixel numbers divided by 18 were used in the model to avoid the conversion issue.

The model shows that p-value of the difference in the Adipocyte Ratio between treatment and control was 0.0278, and therefore we concluded that the Glaciem treatment effect of reducing the adipocyte ratio was statistically significant.

qPCR Biomarker Analysis—In Vivo

The qPCR biomarker analysis was conducted from flash frozen fat tissue from mouse study described above. First, RNA was manually extracted with RNAdvance Tissue kit (Agencourt). RNA was extracted from flash frozen fat tissue according to manufactuer's directions. For example, 5 mm steel beads were placed in the freezer for approximately 15-20 minutes. Next, Qiagen Buffer RLT was cooled to 4° C. for 20 minutes and placed in a 96 well 1.5 mL tube rack for sample transfer. Isopropanol was added to Agencourt wash buffer. 100 mL aliquots of 70% ethanol in water were prepared. The RNA Tissue beads were allowed to warm to 22° C. (˜30 minutes), and then the beads were shaken vigorously and intermittently for at least 15-20 minutes. 1 stainless steel bead was added to a 2 mL round bottom tube stored on dry ice. The tissue sample was then transferred to the 2 mL round bottom tube.

The tube rack was placed on wet ice. Then, the tubes were transferred from dry ice to the rack in wet ice and 350 μL of cold buffer RLT were added to the tubes. The beads were beaten at 30 Hz for 2 minutes. After bead beating, samples were centrifuged for 2 minutes. The supernatant was transferred into 96 well deep well isolation plate.

80 μL of RNATissue beads were combined with 320 μL of isopropanol to create a Bind Buffer. This solution was prepared fresh for each isolation with any unused solution discarded.

400 μL of Bind Buffer was added to the to RLT mixture and slowly mixed. The solution was incubated at room temperature for 10 minutes. Special care was taken to avoid the formation of bubbles while tip mixing. Some bead clumping occurred, but it did not affect the quality or yield.

The mixture was placed on a magnet (96 well plate—Agencourt SPRIPlate) for 6-10 minutes. After the solution became clear, the supernatant was removed from each tube while the tubes remained on the magnet and the solution was discarded.

The tubes were removed from the magnet and the pellets were washed with 800 μL of the Isopropanol Wash Buffer ten times. The plate was placed back on the magnet and, once the solution became clear again, the supernatant was removed from each tube while the tubes remained on the magnet and the solution was discarded.

The plate was removed from the magnet and washed with 600 μL of 70% EtOH. The plate was placed back on the magnet for 10 minutes and, once the solution became clear again, the supernatant was removed from each tube while the tubes remained on the magnet and the solution was discarded. This procedure was repeated with the 70% EtOH one more time. The beads were air dryed 10-20 minutes.

The plate was removed from the magnet again. The beads were eluted with 40 μL of water. The mixture was gently agitated and then incubated 5 minutes at 22° C. The plate was placed on the magnet for 5-10 minutes and/or until the solution became clear. The supernatant was collected and qPCR was collected.

A qPCR panel was run on a QuantStudio 6 Flex machine from Applied Biosystems using TaqMan® Array Fast, 96-well Plate Format 16 from Life Technologies. Fold expression levels for each treatment over control was performed using the double delta Cv method

TABLE 5
qPCR of in vivo samples

Average Fold Formula III vs Placebo

| Biomarker | Formula III | Mirabegron |
|---|---|---|
| Dio2 | 1.8 | 8.3 |
| Cidea | 28.0 | 39.7 |
| Cox7a1 | 5.7 | 27.8 |
| IL6 | 0.8 | 3.3 |
| Tfap2a | 1.2 | 15.2 |
| Prdm16 | 3.5 | 5.0 |
| UCP1 | 73.2 | 170.3 |
| Elovl3 | 37.5 | 19.1 |
| Ppargc1 | 3.0 | 12.0 |
| Cox8b | 19.0 | 38.6 |
| AdipoQ | 4.7 | 13.7 |
| Adrb3 | 18.3 | 16.3 |

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of promoting thermogenesis comprising contacting one or more adipocytes with an activating compound, wherein the activating compound comprises the following structure or salts thereof: 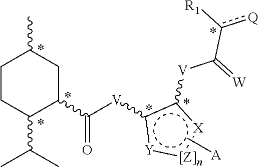
R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and
stereochemistry is variable at the positions marked*.

2. The method of claim 1, wherein the activating compound comprises the following structure or salts thereof: 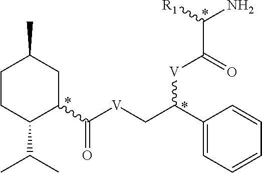
R₁ is H, alkyl, amino alkyl, or alkoxy;
V is —O— or —(NH)—; and
stereochemistry is variable at the positions marked*.

3. The method of claim 1, wherein the activating compound is selected from the group consisting of:

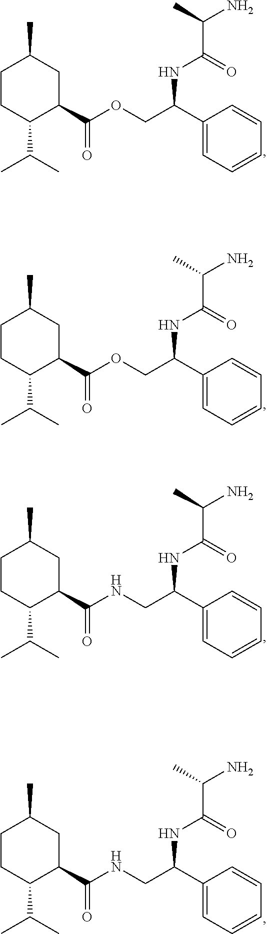 [SMILES: CC(C)[C@@H]1CC[C@@H](C)C[C@H]1C(=O)OC[C@@H](NC(=O)[C@@H](C)N)c1ccccc1 ; CC(C)[C@@H]1CC[C@@H](C)C[C@H]1C(=O)OC[C@@H](NC(=O)[C@H](C)N)c1ccccc1 ; CC(C)[C@@H]1CC[C@@H](C)C[C@H]1C(=O)NC[C@@H](NC(=O)[C@@H](C)N)c1ccccc1 ; CC(C)[C@@H]1CC[C@@H](C)C[C@H]1C(=O)NC[C@@H](NC(=O)[C@H](C)N)c1ccccc1]

4. The method of claim 1, wherein the method further comprises the steps of:
expressing a mitochondrial protein; and
activating one or more adipocytes to induce thermogenesis.

5. The method claim 4, wherein the mitochondrial protein is selected from the group consisting of Ucp1, Ucp2, and combinations thereof.

6. The method of claim 4, wherein the method further comprises activating a receptor upon contact of activating compound with one or more adipocytes.

7. The method of claim 6, wherein the receptor is selected from the group consisting of TrpM8, PPARGC1A, alpha adrenergic receptor, beta adrenergic receptor, and gamma adrenergic receptor.

8. The method of claim 1, wherein one or more adipocytes are present in an affected area.

9. The method of claim 8, wherein the affected area has an excess of adipose tissue.

10. The method of claim 9, wherein the adipose tissue is selected from the group consisting of brown adipocytes, white adipocytes, beige adipocytes, brite adipocytes, subcutaneous adipose tissue, pericardial adipose tissue, marrow adipose tissue, and combinations thereof.

11. The method of claim 8, wherein the treatment reduces the size and quantity of white adipocytes.

12. The method of claim 1, wherein an individual is treated by contacting the activating compound with one or more adipocytes.

13. The method of claim 12, wherein the treatment is selected from the group consisting of the treatment of obesity, the reduction of adipose tissue, body contouring, body shaping, type 1 diabetes, type 2 diabetes, insulin-resistance, dyslipidemia, irritable bowel syndrome, chronic pain, neuropathic pain, and inflammatory pain.

14. The method of claim 13, wherein the activating compound is contacted with one or more adipocytes through a route selected from the group consisting of injection, buccal, enteral, inhalable, infused, intramuscular, intrathecal, intravenous, nasal, ophthalmic, oral, otic, rectal, subcutaneous, sublingual, topical, transdermal, and combinations thereof.

15. The method of claim 14, wherein the activating compound is contacted with one or more adipocytes in a form selected from the group consisting of tablet, pill, suppository, micro-needle patch, transdermal patch, suspension, solution, body wrap, and combinations thereof.

16. A method of treatment comprising contacting one or more adipocytes with an activating compound, wherein the activating compound comprises the following structure or salts thereof: 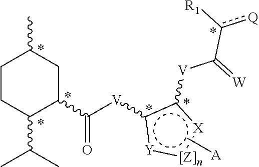
R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and
stereochemistry is variable at the positions marked*.

17. A device comprising
a therapeutically effective amount of an activating compound and
a means for contacting one or more adipocytes with the activating compound.

18. The device of claim 17, wherein the activating compound comprises the following structure: 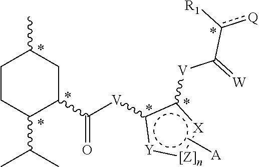
R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≥1 and Z is selected from aliphatic CH₂, aromatic CH, or heteroatom;
A=lower alkoxy, lower alkylthio, aryl, substituted aryl or fused aryl; and
stereochemistry is variable at the positions marked*.

19. The device of claim 17, wherein the activating compound comprises the following structure or salts thereof: 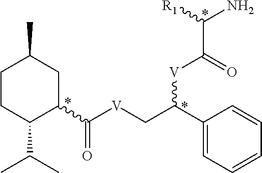
R₁ is H, alkyl, amino alkyl, or alkoxy;
V is —O— or —(NH)—; and
stereochemistry is variable at the positions marked*.

20. The device of claim 17, wherein the means for contacting the activating compound with one or more adipocytes is selected from the group consisting of injection, buccal, enteral, inhalable, infused, intramuscular, intrathecal, intravenous, nasal, ophthalmic, oral, otic, rectal, subcutaneous, sublingual, topical, transdermal, and combinations thereof.


SEQUENCE LISTING (1 sequence)

SEQ ID NO: 1 - DNA, 3315 bp, Homo Sapiens
atgtccttcgagggagccaggctcagcatgaggagccgcagaaatggtactatgggcagcacccggaccctgtactccagtgtatctcggagcacagacgtgtcctacagtgacagtgatttggtgaattttattcaggcaaattttaaaaaacgagaatgtgtcttctttaccagagactccaaggccatggagaacatatgcaagtgtggttatgcccagagccagcacatcgaaggcacccagatcaaccaaaatgagaagtggaactacaaaaaacataccaaggagtttccaacagacgccttcggggacattcagtttgagactctggggaagaaaggcaagtacttacgcttgtcctgtgacaccgactctgaaactctctacgaactgctgacccagcactggcacctcaaaacacccaacctggtcatttcagtgacgggtggagccaaaaactttgctttgaagccacgcatgcgcaagatcttcagcaggctgatttacatcgcacagtctaaaggtgcgtggattctcactggaggcactcactacggcctgatgaagtacataggcgaggtggtgagagacaacaccatcagcaggaactcagaagagaacatcgtggccattggcatcgcagcatggggcatggtctccaacagggacaccctcatcaggagctgtgatgatgagggacatttttcagctcaatacatcatggatgactttaccagagaccctctatacatcctggacaacaaccatacccacctgctgcttgtggacaacggttgtcatggacaccccacagtggaagccaagctccggaatcagctggaaaagtacatctctgagcgcaccagtcaagattccaactatggtggtaagatccccatcgtgtgttttgcccaaggaggtggaagagagactctaaaagccatcaacacctctgtcaaaagcaagatcccttgtgtggtggtggaaggctcggggcagattgctgatgtgatcgccagcctggtggaggtggaggatgttttaacctcttccatggtcaaagagaagctggtacgctttttaccacgcactgtgtcccggctgcctgaagaggaaattgagagctggatcaaatggctcaaagaaattcttgagagttctcacctactcacagtaattaagatggaagaggctggagatgagattgtgagcaacgccatttcctatgcgctgtacaaagccttcagcactaatgagcaagacaaggacaactggaatggacagctgaagcttctgctggagtggaaccagttggaccttgccagtgatgagatcttcaccaatgatcgccgctgggagtctgccgaccttcaggaggtcatgttcacggctctcataaaggacagacccaagtttgtccgcctctttctggagaatggcctgaatctgcagaagtttctcaccaatgaagtcctcacagagctcttctccacccacttcagcaccctagtgtaccggaatctgcagatcgccaagaactcctacaatgacgcactcctcacctttgtctggaagttggtggcaaacttccgtcgaagcttctggaaagaggacagaagcagcagggaggacttggatgtggaactccatgatgcatctctcaccacccggcacccgctgcaagctctcttcatctgggccattcttcagaacaagaaggaactctccaaggtcatttgggagcagaccaaaggctgtactctggcagccttgggggccagcaagcttctgaagaccctggccaaagttaagaatgatatcaacgctgctggggaatcggaggaactggccaatgaatatgagacccgagcagtggagttgttcaccgagtgttacagcaatgatgaagacttggcagaacagctactggtctactcctgcgaagcctggggtgggagcaactgtctggagctggcagtggaggctacagatcagcatttcatcgctcagcctggggtccagaatttcctttctaagcaatggtatggagagatttcccgagacacgaagaactggaagattatcctgtgtctattcatcatccccttagtgggctgtggcctcgtatcatttaggaagaaacccattgacaagcacaagaagctgctgtggtactatgtggccttcttcacgtcgcccttcgtggtcttctcctggaacgtggtcttctacatcgccttcctcctgctgtttgcctatgtgctgctcatggacttccactcagtgccacacacccccgagctgatcctctacgccctggtcttcgtcctcttctgtgatgaagtgaggcagtggtacatgaacggagtgaattatttcaccgacctatggaacgttatggacaccctgggactcttctacttcatagcgggtattgtattccggctccactcttctaataaaagctcgttgtactctgggcgcgtcattttctgtctggattacattatattcacgctaaggctcatccacattttcaccgtcagcaggaacttgggacccaagattataatgctgcagcggatgctgatcgacgttttcttcttcctgttcctctttgctgtgtggatggtggcctttggcgtggccagacaggggatcctaaggcaaaatgaacagcgctggagatggatcttccgctctgtcatctatgagccctacctggccatgtttggccaggttcccagtgacgtggatagtaccacatatgacttctcccactgtaccttctcgggaaatgagtccaagccactgtgtgtggagctggatgagcacaacctgccccgcttccctgagtggatcaccattccgctggtgtgcatctacatgctctccaccaatatccttctggtcaacctcctggtcgccatgtttggctacacggtaggcattgtacaggagaacaacgaccaggtctggaaattccagcggtacttcctggtgcaggagtactgcaaccgcctaaacatccccttccccttcgttgtcttcgcttatttctacatggtggtgaagaagtgtttcaaatgctgctgtaaagagaagaatatggagtctaatgcctgctgtttcagaaatgaggacaatgagactttggcgtgggagggtgtcatgaaggagaattaccttgtcaagatcaacacgaaagccaacgacaactcagaggagatgaggcatcggtttagacaactggactcaaagcttaacgacctcaaaagtcttctgaaagagattgctaataacatcaagtaa